(19) United States
(12) Patent Application Publication
(10) Pub. No.: US 2018/0340622 A1
(43) Pub. Date: Nov. 29, 2018

(54) Plug and Stem Check-Valve

(71) Applicant: Andrew Kyle Godfrey, New Albany, IN (US)

(72) Inventor: Andrew Kyle Godfrey, New Albany, IN (US)

(73) Assignee: Andrew Kyle Godfrey, New Albany, IN (US)

(21) Appl. No.: 15/993,030

(22) Filed: May 30, 2018

Related U.S. Application Data
(60) Provisional application No. 62/511,987, filed on May 27, 2017.
(60) Provisional application No. 62/511,983, filed on May 27, 2017.

(51) Int. Cl. F16K 15/06; F16K 15/02; A45C 11/00; B65D 47/24

(52) U.S. Cl. CPC F16K 15/06; F16K 15/021; A61L 12/086; B65D 47/248; A45C 11/005

(57) ABSTRACT

A one way plug and stem check-valve is disclosed. The one way plug and stem check-valve includes a plug that has a plug base of a smaller size than a plug top wherein the plug base and the plug top are about parallel, the plug having a sidewall connecting the plug top and the plug base together with a central axis connecting the plug base and the plug top at their respective centers, a leg wherein two or more of said legs are flexibly coupled to the plug base at an angle of about 1 degree to 45 degrees diverging away from the central axis, wherein the two or more legs are distributed about the radius of the plug base, a housing that encapsulates the plug with openings at the plug top and the plug base with similar geometry to that of the sidewall of the plug wherein the sidewall of the plug creates a fluid seal with an inside wall of the housing while in a static position, the housing allows displacement of the plug and legs along the central axis in the direction of the top of the plug wherein the said legs absorb energy as they are displaced inward toward the central axis of the plug by the housing, this absorbed energy is expelled by the legs as the legs and the plug return to their static position.

### TECHNICAL FIELD

The present application relates to fluid flow that is regulated by a plug and stem check-valve and applications of said plug and stem check-valve.

### BACKGROUND

Standard contact lens cases are a simple and effective product, but provide little utility to the user other than simply storing contact lenses. There is room for improvement to reduce the amount of time required to store and properly clean contact lens. There is also room for improvement on the portability of contact lens storing and cleaning paraphernalia.

Therefore there is an unmet need for a more convenient, more portable, and sanitary solution for cleaning, storing, and transporting contact lenses.

There are currently no solutions on the market for a self-resisting, single piece one-way valve. This valve provides a function that can be mimicked by other valve designs (restricting fluid flow to a single direction) but does it in such a manner that is completely novel and is an excellent alternative to many currently existing one way valves.

Therefore there is an unmet need for a cheaper and simpler to produce one-way fluid flow valve.

### SUMMARY

A one way plug and stem check-valve is disclosed. The one way plug and stem check-valve includes a plug that has a plug base of a smaller size than a plug top wherein the plug base and the plug top are about parallel, the plug having a sidewall connecting the plug top and the plug base together with a central axis connecting the plug base and the plug top at their respective centers, a leg wherein two or more of said legs are flexibly coupled to the plug base at an angle of about 1 degree to 45 degrees diverging away from the central axis, wherein the two or more legs are distributed about the radius of the plug base, a housing that encapsulates the plug with openings at the plug top and the plug base with similar geometry to that of the sidewall of the plug wherein the sidewall of the plug creates a fluid seal with an inside wall of the housing while in a static position, the housing allows displacement of the plug and legs along the central axis in the direction of the top of the plug wherein the said legs absorb energy as they are displaced inward toward the central axis of the plug by the housing, this absorbed energy is expelled by the legs as the legs and the plug return to their static position.

### BRIEF DESCRIPTION OF DRAWINGS

FIG. 1 is a closed plug and stem check-valve apparatus.

FIG. 2 is an open plug and stem check-valve apparatus.

FIG. 3 is a closed plug and stem check-valve apparatus.

FIG. 4 is an open plug and stem check-valve apparatus.

FIG. 5 is a closed plug and stem check-valve apparatus.

FIG. 6 is an open plug and stem check-valve apparatus in a negative flow position.

FIG. 7 is an open plug and stem check-valve apparatus in a positive flow position.

FIG. 8 is stem valve with a plug, two legs, and two corresponding notches.

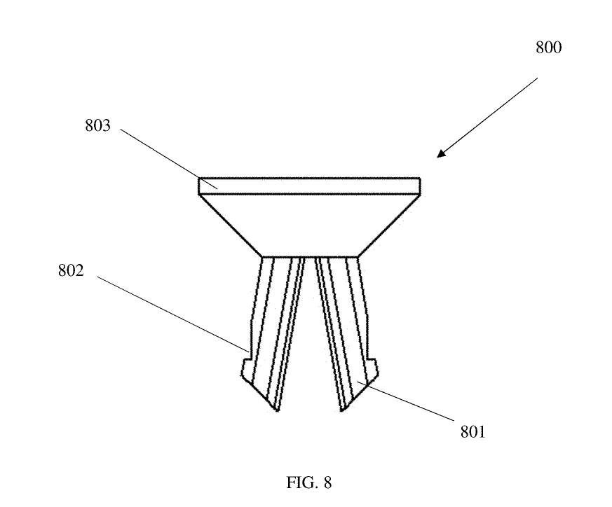

FIG. 9 is stem valve with a plug, a conical wedge, and a corresponding ledge.

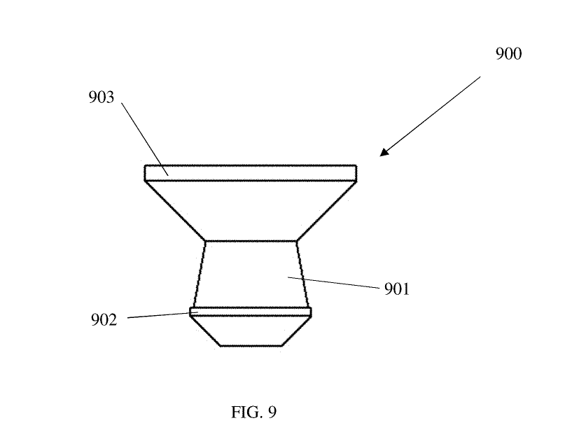

FIG. 10 is stem valve with a plug and incremented concave legs.

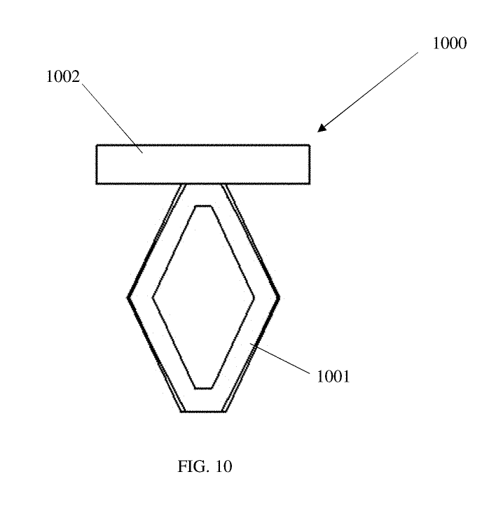

FIG. 11 is stem valve with a plug and curved concave legs.

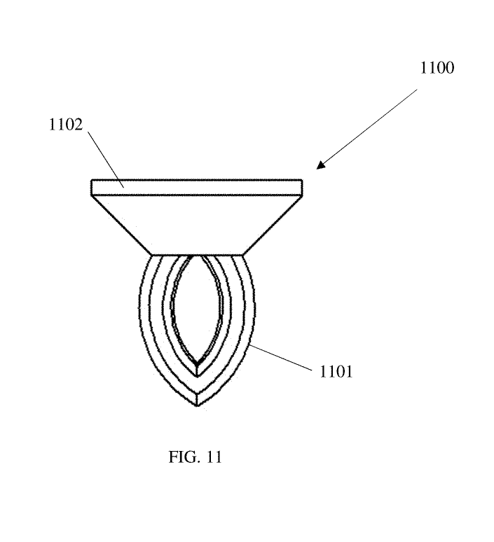

FIG. 12 is stem valve with a plug and curved convex legs.

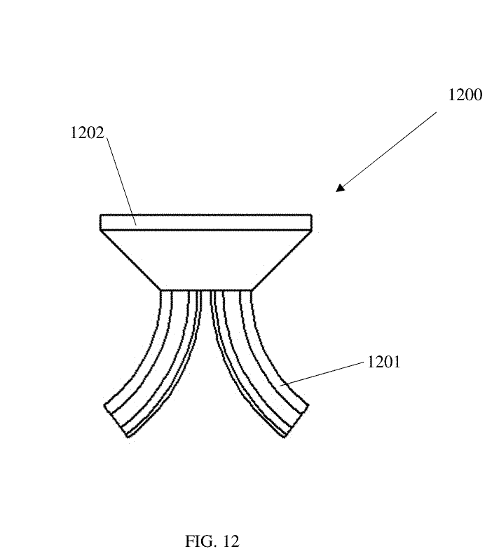

FIG. 13 is a self-filling contact lens case with three one way valves and a reusable tank.

FIG. 14 is a self-filling contact lens case with three one way valves, a fluid spout, and a disposable tank.

### DETAILED DESCRIPTION

Static position is defined as the position that the valve rests at when no outside forces are applied to the valve, this can be seen in 100, 300, 500.

An open position is defined as the position where the valve interior is forced in the direction of the positive pressure gradient as seen in 200, 400, 600, 700.

A check valve is defined as a fluid flow valve that prevents flow of fluid in an undesired direction as shown in 1302, 1404.

A valve stem is defined as a plug that seals off flow between two different areas along with corresponding base (either legs, or a conical wedge), this can be seen in 103, 203, 304, 404, 503, 603, 703, 800, 900, 1000, 1100, 1200.

A notch 802 is defined as a section of material either added or removed from an otherwise contiguous surface. Notches in this application are used to prevent undesirable/excessive displacement in the described valves, while still allowing the valve to open enough to allow reasonable fluid flow.

A slot is defined as a section of material removed from an otherwise contiguous surface to enable proper fluid flow can be seen on the legs shown in 303, 403.

A contact lens case 1300, 1400 is an apparatus that houses one or more contact lens storage bowls 1303, 1402 and may also be coupled to a reservoir. The contact lens case and the reservoir may be secured together by a variety of techniques including but not exclusive to: friction, clips, ties, or gravity.

A reservoir 1301, 1401 is a fluid storage tank that may be connected and removed with the contact lens case. When the reservoir and the contact lens case are coupled the fluid in the reservoir may travel to the contact lens bowl(s) at the activation force of the user.

A directional fluid flow control device 101, 201, 301, 401, 501, 601, 701 is a nozzle coupled to the valve apparatus that yields a desirable behavior of fluids being expelled from the valve. The geometry of this device may be, but is not limited to: conical (shown in drawings), elliptical, and cylindrical geometries. Shapes such as these may be modified by a person having ordinary skill in the art to produce a desired fluid discharge volume and shape.

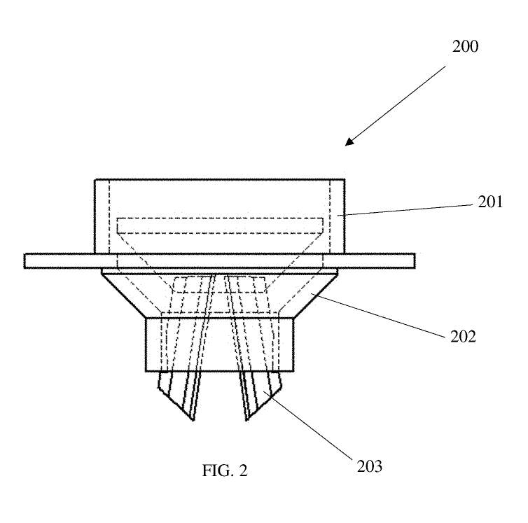

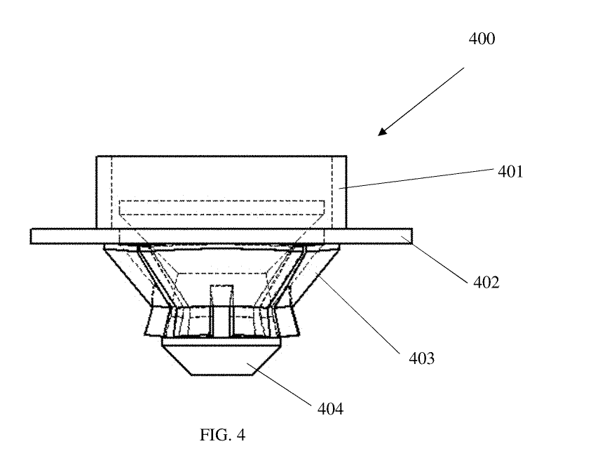

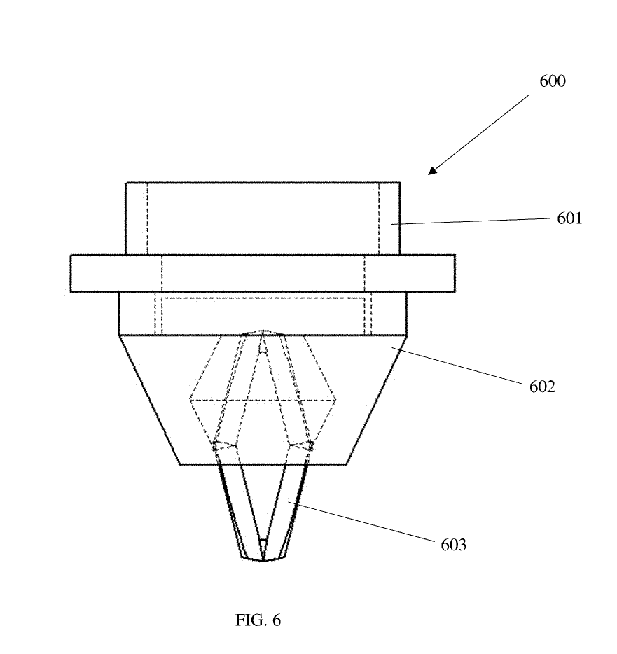

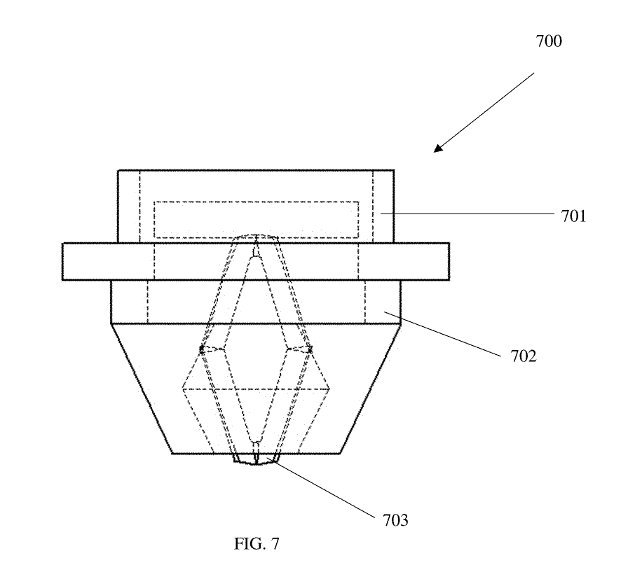

Absorbed energy is defined as kinetic energy that is transferred to legs of the valve apparatus. This transfer occurs when a pressure differential acts to force the valve from its static position, to an open position which can be seen in FIGS. 2, 4, 6, 7.

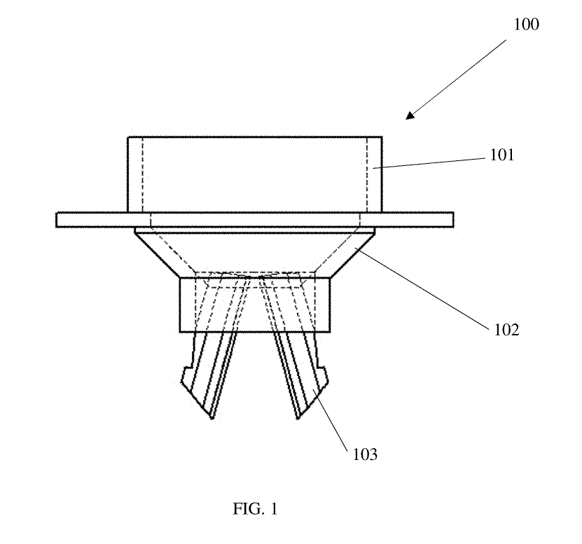

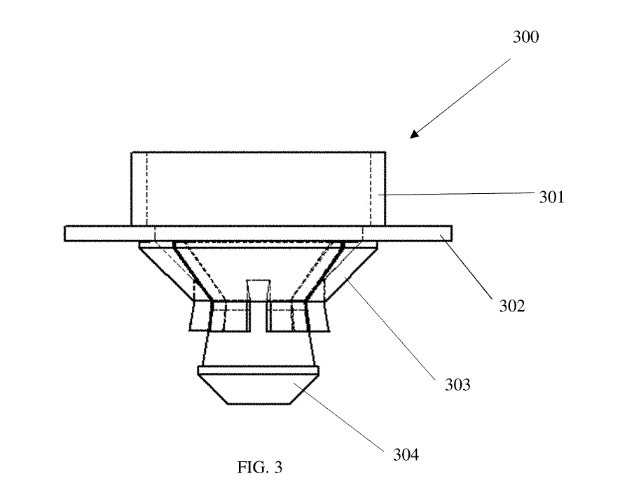

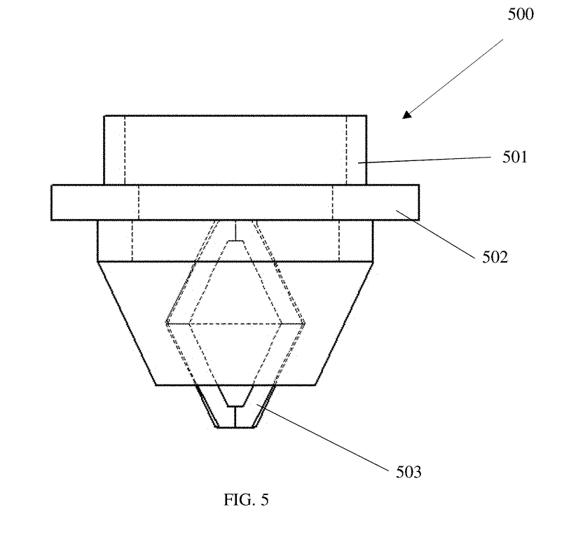

Expelled energy is defined as the energy that is converted from absorbed energy back to kinetic energy. This expelled energy is used to return the valve to its static position from an open position which can be seen in FIGS. 1, 3, 5.

Inventions disclosed in this application may be manufactured by various well known manufacturing techniques including but not limited to: mold injection, forging, additive manufacturing, casting and milling.

For the valve to be assembled after the individual parts have been manufactured, the inside part of the valve need only be pressed into the corresponding housing. The flexible nature of the legs of the valve apparatus (either the legs attached to the plug, or the legs attached to the housing) will allow for proper assembly.

The disclosed inventions may be used at various different proportions and for various fluids of different viscosity. A person having ordinary skill in the art would determine the resistant force required in the valve apparatus and adjust geometry and material choices appropriately.

One of the benefits of the disclosed valve designs is the prevention of cross contamination between the reservoir and whatever may be on the other side of the valve. Many standard valves have cavities that are exposed to the other side of the valve. These cavities can easily house undesired contaminated fluid that can potentially contaminate otherwise clean fluids. The disclosed valve designs perform the task of restricting fluid flow with a small number of, and easy to manufacture parts. The shape of the plug prevents fluid from being squirted out of the contact lens bowl, it diverts the fluid toward the walls of the contact lens bowl.

These disclosed valve designs only require two parts, the inside part, and the housing.

A ledge 902 is defined as a surface that prevents undesirable displacement of the valve stem.

The valve may be made of a myriad of different materials including, but not exclusive to: plastics, metals, composites, and ceramics. The only requirement is that the legs 303, 403, 801, 1001, 1101, 1201 must be flexible enough to allow the valve stem to move under the intended pressure differential. The plug 803, 903, 1002, 1102, 1202 and the housing 102, 202, 302, 402, 502, 602, 702 that comes in contact with the plug must be made of such materials that enable an about fluid tight seal. Additional materials may be added to the surface of the plug and or the surface the plug comes in contact with to ensure a good seal, some of these materials include but are not limited to: silicone, rubber, paper, cardboard, and textured plastics.

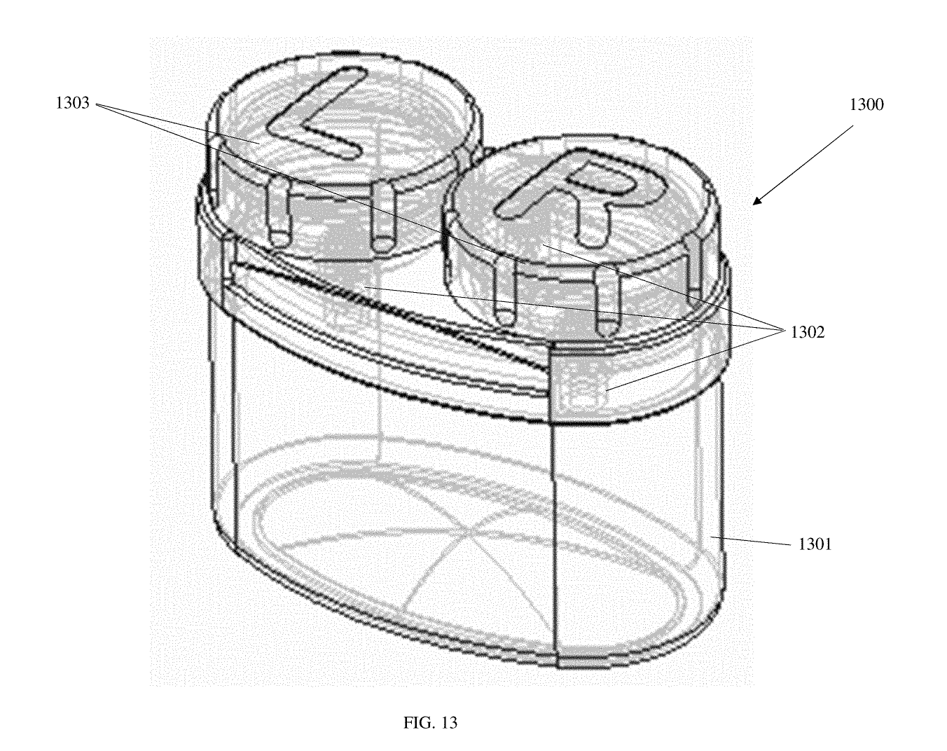

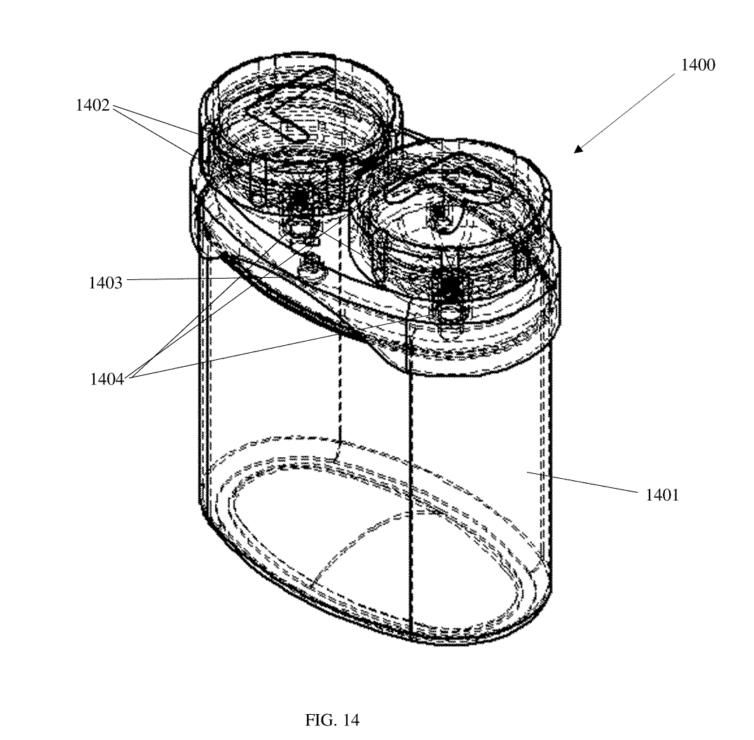

The reservoir may be reusable shown in FIG. 13, or the reservoir may be disposable as shown in FIG. 14. Disposable and reusable reservoirs pose different potential benefits to the user.

A fluid spout 1403 may be incorporated into the design of the reservoir. This fluid spout allows the user to use the fluid in the reservoir for more than just refilling their contact lens case. This spout may be closed with a cap, this cap may be used with the contact case.

The reservoir may have self-sealing valves that connect to the valves of the contact case. These valves will close themselves off when the reservoir and the contact case are separated. These valves will open when they are connected with the contact case.

The reservoir may be made of a flexible material that allows it to be deformed when sustaining force. This deformation will reduce the internal volume of the reservoir and force the fluid either into the contact lens case, or out of the fluid spout.

There are many ways the fluid may be displaced from the reservoir. The reservoir may contain a small pump that can be operated by the user to displace the fluid. The reservoir may be designed to be collapsible, this way it could be deformed to displace the fluid and not require an air valve to replace the displaced fluid.

An air valve may be connected to directly to the reservoir to allow air to replenish any displaced fluid. This valve allows the reservoir to maintain its original volume by replacing any lost fluid.

The air valve may alternatively be mounted on the contact lens case and connect with the reservoir when the two bodies are united.

A cap is a device that is screwed onto the top of the contact lens bowl. The cap prevents any fluid from escaping the contact lens bowl.

For a user to use the apparatuses shown in FIGS. 13, 14 the user would remove the desired cap(s), dump out any old fluids, then squeeze the reservoir to fill the contact lens bowl(s) with fluid from the reservoir.

The check valves connected to the contact lens bowls may have tubes connected to them that extend into the bottom of the reservoir. Alternatively these tubes may be connected to the self-sealing valves of the reservoir.

For a user to use the fluid spout in FIG. 14, the user would separate the reservoir from the contact case, then use the reservoir as a simple squeeze bottle.

### PARENT CASE TEXT

In accordance with 37 C.F.R. .sctn.1.76, a claim of priority is included in an Application Data Sheet filed concurrently herewith. Accordingly, the present invention claims priority under 35 U.S.C. .sctn..sctn.119(e), 120, 121, and/or 365(c) to U.S. Provisional Patent Application No. 62/511,987, entitled “One-Way Valve”, filed on May 27, 2017 and to U.S. Provisional Patent Application No. 62/511,983, entitled “Self-Filling Contact Lens Case”, filed on May 27, 2017. The contents of which the above referenced application is incorporated herein by reference in its entirety.

1. A one way fluid flow check-valve, comprising:
a plug that has a plug base of a smaller size than a plug top wherein the plug base and the plug top are about parallel, the plug having a sidewall connecting the plug top and the plug base together with a central axis connecting the plug base and the plug top at their respective centers
a leg wherein two or more of said legs are flexibly coupled to the plug base at an angle of about 1 degree to 45 degrees diverging away from the central axis. Wherein the two or more legs are distributed about the radius of the plug base
a housing that encapsulates the plug with openings at the plug top and the plug base with similar geometry to that of the sidewall of the plug wherein the sidewall of the plug creates a fluid seal with an inside wall of the housing while in a static position, the housing allows displacement of the plug and legs along the central axis in the direction of the top of the plug wherein the said legs absorb energy as they are displaced inward toward the central axis of the plug by the housing, this absorbed energy is expelled by the legs as the legs and the plug return to their static position.

2. The plug and leg of claim 1 wherein the plug and leg are made of a continuous, solid, flexible material.

3. The leg of claim 1 wherein a notch is added to the exterior of about the outermost location of the bottom of the leg

4. The leg of claim 1 wherein the leg is concavely curved with respect to the central axis.

5. The leg of claim 1 wherein the leg is convexly curved with respect to the central axis.

6. The housing of claim 1 wherein a directional fluid flow control device is rigidly coupled to said housing.

7. A one way fluid flow check-valve, comprising:
a plug that has a plug base of a smaller size than a plug top wherein the plug base and the plug top are about parallel, the plug having a sidewall connecting the plug top and the plug base together with a central axis connecting the plug base and the plug top at their respective centers
a conical wedge having a conical wedge top of about equal radius to that of the plug base wherein said conical wedge top is rigidly coupled to the plug base
the conical wedge having a conical wedge bottom with a radius greater than the conical wedge top and having a radius less than that of the plug top wherein the angle between the conical wedge bottom and the conical wedge top is about 1 to 45 degrees
the conical wedge having a sidewall connecting the conical wedge top and the conical wedge base together with the central axis connecting the conical wedge top and the conical wedge base at their respective centers
a housing and a housing leg wherein 2 or more housing legs and the housing encapsulate the plug with openings at the top of the plug and the base of the plug with similar geometry to that of the sidewall of the plug wherein the sidewall of the plug creates a fluid seal with an inside wall of the housing while in a static position, the 2 or more said housing legs are flexibly coupled to the bottom of the housing wherein the 2 or more housing legs are distributed about the radius of the plug and the conical wedge, the housing and housing legs allow displacement of the plug and conical wedge along the central axis in the direction of the top of the plug wherein the said housing legs absorb energy as they are displaced away from the central axis of the plug, this absorbed energy is expelled by the housing legs as the housing legs and the plug return to their static position.

8. The plug and conical wedge of claim 7 wherein the plug and conical wedge are made of a continuous, solid material.

9. The housing leg of claim 7 wherein the housing leg is made of a continuous, solid, flexible material.

10. The conical wedge of claim 7 wherein a notch is added to the exterior of about the outermost location of the bottom of the leg.

11. The conical wedge of claim 7 wherein the conical wedge is concavely curved with respect to the central axis.

12. The conical wedge of claim 7 wherein the conical wedge is convexly curved with respect to the central axis.

13. The housing leg of claim 7 wherein a slot is cut into the housing leg.

14. A two way fluid flow check-valve, comprising:
a plug that has a plug base of a about equal size to that of a plug top wherein the plug base and the plug top are about parallel, the plug having a sidewall connecting the plug top and the plug base together with a central axis connecting the plug base and the plug top at their respective centers
a leg wherein two or more of said legs are flexibly coupled to the plug base at an angle of about 1 degree to 45 degrees diverging away from the central axis, said legs then converge back together at a point on the central axis at an angle of about 1 degree to 45 degrees wherein the two or more legs are distributed about the radius of the plug base
a housing that encapsulates the plug with openings at the plug top and the plug base with similar geometry to that of the sidewall of the plug wherein the sidewall of the plug creates a fluid seal with an inside wall of the housing while in a static position, the housing allows displacement of the plug and legs along the central axis in the direction of the top of the plug, and the base of the plug wherein the said legs absorb energy as they are displaced inward toward the central axis of the plug by the housing, this absorbed energy is expelled by the legs as the legs and the plug return to their static position.

15. The plug and leg of claim 14 wherein the plug and leg are made of a continuous, solid, flexible material.

16. The leg of claim 14 wherein two or more legs are flexibly coupled together

17. The leg of claim 14 wherein a notch is added to the bottom of the leg

18. The leg of claim 14 wherein a notch is added to the housing

19. The leg of claim 14 wherein the leg is concavely curved with respect to the central axis.

20. The housing of claim 14 wherein a directional fluid flow control device is rigidly coupled to said housing.
